(19) United States
(12) Patent Application Publication
(10) Pub. No.: US 2018/0028149 A1
(43) Pub. Date: Feb. 1, 2018

(54) FLEXIBLE SURFACE SENSING APPARATUS FOR SOFT AND FLEXIBLE SURFACE

(71) Applicant: MiiCs & Partners (Shenzhen) Co., Ltd., Shenzhen (CN)

(72) Inventor: JUAN WANG, Shenzhen (CN)

(21) Appl. No.: 15/652,633

(22) Filed: Jul. 18, 2017

(30) Foreign Application Priority Data
Jul. 26, 2016 (CN) 201610593605.5

(51) Int. Cl. A61B 8/00; H05K 1/02; A61B 8/04; H01L 23/538; B06B 1/06; A61B 8/02; H05K 1/14; H01L 27/12

(52) U.S. Cl. CPC A61B 8/4227; H05K 1/14; H05K 1/028; H01L 27/1218; H01L 23/5387; B06B 1/06; A61B 8/02; A61B 8/04; A61B 8/4236; A61B 8/4494; H05K 2201/10151; H05K 2201/10166

(57) ABSTRACT

A flexible sensing apparatus for soft and curving objects which can sense vital signals of the specified object and convert reflected signals into electrical signals. The flexible sensing apparatus includes a transmitting layer, a readout layer, and a receiving layer, arranged in that order. The transmitting layer generates ultrasonic waves. The receiving layer receives reflected ultrasonic waves and converts them into localized electric charges. The readout layer converts the localized electric charges into electrical signals to represent detected vital signs. The receiving layer is adjacent to the specified object, and the transmitting layer faces away from the specified object. The readout layer comprises a flexible thin film transistor (TFT) array. The flexible TFT array converts the localized electric charges generated by different positions of the third conductive structure.

### CROSS-REFERENCE TO RELATED APPLICATIONS

This application claims priority to Chinese Patent Application No. 201610593605.5 filed on Jun. 26, 2016, the contents of which are incorporated by reference herein.

### FIELD

The subject matter herein generally relates to a flexible sensing apparatus, especially to an ultrasonic sensing patch.

### BACKGROUND

Sensing apparatuses are widely used. Reflective type apparatus typically cooperates with a mobile phone or a watch to sense heart rates. A complementary metal-oxide-semiconductor (CMOS) sensor is used in the reflective type sensing apparatus for detecting heart rates. The CMOS sensor senses a light intensity difference based on a vasoconstriction, and transmits an output signal. The output signal of the CMOS sensor is calculated by a method of photo plethsmography (PPG) to obtain heart rates. However, the sensing apparatus is large in size, and adhesion of the sensing apparatus is poor. Thus, there is room for improvement in the art.

### BRIEF DESCRIPTION OF THE FIGURES

Implementations of the present technology will now be described, by way of example only, with reference to the attached figures.

FIG. 1 is an isometric view of a first exemplary embodiment of a flexible sensing apparatus, the flexible sensing apparatus comprising a readout layer.

FIG. 2 is a planar view of the readout layer of FIG. 1.

FIG. 3 is an isometric view of the flexible sensing apparatus applied to a specified object.

FIG. 4 is an isometric view of a second exemplary embodiment of a flexible sensing apparatus, the flexible sensing apparatus comprising a readout layer.

FIG. 5 is a planar view of the readout layer of FIG. 4.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the exemplary embodiments described herein.

The term “substantially” is defined to be essentially conforming to the particular dimension, shape, or other feature that the term modifies, such that the component need not be exact. For example, “substantially cylindrical” means that the specified object resembles a cylinder, but can have one or more deviations from a true cylinder. The term “comprising” means “including, but not necessarily limited to”; it specifically indicates open-ended inclusion or membership in a so-described combination, group, series, and the like.

The present disclosure is described in relation to a flexible sensing apparatus, which may cooperate with other apparatus for detecting physiological parameters of specified objects. The specified object may be humans or animals. The flexible sensing apparatus may generate sensing signals. The flexible sensing apparatus may be integrated with the other apparatus, or used separately. The other apparatus may be, but is not limited to, a mobile device, a watch, a computer device, or any other apparatus which is capable of processing the sensing signals and generating a viewable result.

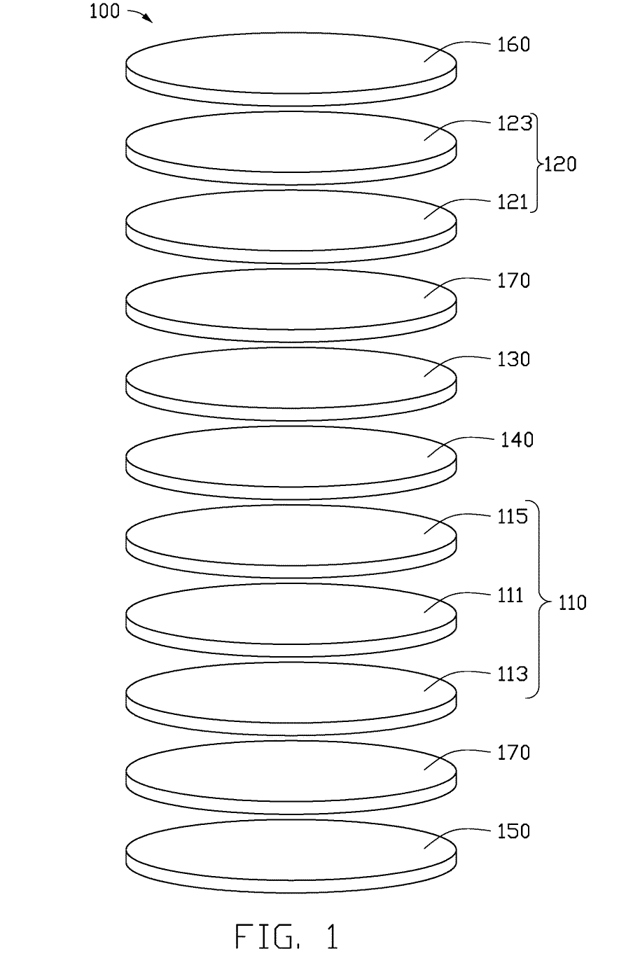

FIG. 1 illustrates a first exemplary embodiment of a flexible sensing apparatus 100 applied on a curved surface or a rough surface. The flexible sensing apparatus 100 senses vital signs related to blood, for example, a heart rate, a pulse, a blood pressure, and so on. The flexible sensing apparatus 100 generates ultrasonic waves, receives the ultrasonic waves reflected by the specified object, and generates electrical signals representing the vital signs. In at least one exemplary embodiment, the flexible sensing apparatus 100 is a flexible sensing patch.

The flexible sensing apparatus 100 includes a transmitting layer 110, a receiving layer 120, a readout layer 130, a first flexible circuit board 140, a second flexible circuit board 150, a third flexible circuit board 160, and two adhesive layers 170. The transmitting layer 110 generates the ultrasonic waves. The receiving layer 120 converts the ultrasonic waves reflected by the specified object into localized electric charges, and transmits the localized electric charges to the readout layer 130. The readout layer 130 converts the localized electric charges into electrical signals and outputs to an external circuit (not shown). The first flexible circuit board 140 provides a first specified voltage to the transmitting layer 110. The second flexible circuit board 150 provides a second specified voltage to the transmitting layer 110. The third flexible circuit board 160 transmits data to the receiving layer 120. One of the adhesive layers 170 is attached to the transmitting layer 110 and the second flexible circuit board 150 together, and another of the adhesive layers 170 is attached to the receiving layer 120 and the readout layer 130 together. In other exemplary embodiments, the flexible sensing apparatus 100 can include another adhesive layer for pasting the flexible sensing apparatus 100 on the specified object.

The transmitting layer 110 includes a transmitting element 111, a first conductive structure 113, and a second conductive structure 115. The transmitting element 111 is between the first conductive structure 113 and the second conductive structure 115. The first conductive structure 113 is between the transmitting element 111 and the second flexible circuit board 150. The second conductive structure 115 is between the transmitting element 111 and the first flexible circuit board 140. The first specified voltage is applied by the first conductive structure 113, and the second specified voltage is applied by the second conductive structure 115, a voltage difference between the first conductive structure 113 and the second conductive structure 115 drives the transmitting element 111 to generate the ultrasonic waves. In at least one exemplary embodiment, the transmitting element 111 is made of a piezoelectric material, for example, polyvinylidene fluoride (PVDF), BaiO₃, PbiO₃, Pb(Zri)O₃, plumbum scandium tantalite (PST), quartz, (Pb, Sm)iO₃, PMN(Pb(MgNb)O₃)—PT(PbiO₃), and PVDF-TrFE. In at least one exemplary embodiment, the first conductive structure 113 and the second conductive structure 115 are substantially circular or elliptical in shape. In other exemplary embodiments, the first conductive structure 113 and the second conductive structure 115 can include a plurality of sensing electrodes separated from each other. Each of the sensing electrodes can be rectangular, a wave shape, or a serrated shape, not to be limited in shape. The first conductive structure 113 and the second conductive structure 115 may be formed on a surface of the transmitting element 111 by a vacuum sputtering manner, a painting manner, or a coated manner. In at least one exemplary embodiment, opposite surfaces of the transmitting element 111 can attach to the first conductive structure 113 and the second conductive structure 115 respectively by a conductive adhesive structure.

The receiving layer 120 includes a receiving element 121 and a third conductive structure 123. The receiving element 121 is between the third conductive structure 123 and the adhesive layer 170. The receiving element 121 can receive the ultrasonic waves reflected by the specified object. The third conductive structure 123 is between the receiving element 121 and the third flexible circuit board 160. The receiving element 121 can convert the reflected ultrasonic waves into localized electric charges. The third conductive structure 123 may control the localized electric charges of the receiving element 121 to be transmitted to the readout layer 130. In at least one exemplary embodiment, strength of the reflected ultrasonic waves depends on a vasoconstriction within the specified object. In at least one exemplary embodiment, the receiving element 121 is made of piezoelectric material, for example, polyvinylidene fluoride (PVDF), BaiO₃, PbiO₃, Pb(Zri)O₃, plumbum scandium tantalite (PST), quartz, (Pb, Sm)iO₃, PMN(Pb(MgNb)O₃)—PT(PbiO₃), and PVDF-TrFE. In at least one exemplary embodiment, the third conductive structure 123 is a bias electrode layer, and is substantially a plane. In other exemplary embodiments, the third conductive structure 123 can include sensing electrodes separated from each other, each of the sensing electrodes can be rectangular, wave shaped, or a serrated shape, but not to be limited thereto. The third conductive structure 123 can be formed on a surface of the receiving element 121 by a vacuum sputtering manner, a painting manner, or a coated manner. In at least one exemplary embodiment, the receiving element 121 can be pasted to the third conductive structure 123 by a conductive adhesive structure.

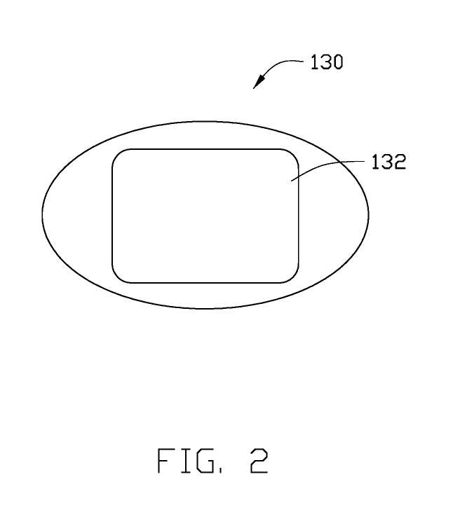

Referring to FIG. 2, the readout layer 130 can convert the localized electric charges transmitted by the adhesive layer 170 into electrical signals, and transmit the electrical signals to a readout circuit (not shown) for calculating a vital sign. The readout layer 130 includes a one-piece readout unit 132. The readout unit 132 includes a flexible thin film transistor (TFT) array, which receives the localized electric charges. The readout unit 132 is substantially rectangular. In at least one exemplary embodiment, the flexible TFT array can be high temperature polycrystalline TFT (HTPS-TFT), low temperature polycrystalline TFT (LTPS-TFT), amorphous silicon TFT (a-Si-TFT), or indium gallium zinc oxide TFT (IGZO TFT). In other exemplary embodiments, the readout unit 132 can be a triangle shape, an annular shape, or a polygon shape.

The first flexible circuit board 140 and the second flexible circuit board 150 are electrically connected to the first conductive structure 113 and the second conductive structure 115 respectively. The first flexible circuit board 140 provides the first specified voltage to the second conductive structure 115, and the second flexible circuit board 150 provides the second specified voltage to the first conductive structure 113 The second specified voltage is different from the first specified voltage. The third flexible circuit 160 transmits data to the third conductive structure 123. In at least one exemplary embodiment, the first flexible circuit board 140, the second flexible circuit board 150, and the third flexible circuit board 160 are insulated from each other. In other exemplary embodiments, the first flexible circuit board 140, the second flexible circuit board 150, and the third flexible circuit board 160 may be electrically connected, and some of the first flexible circuit board 140, the second flexible circuit board 150, and the third flexible circuit board 160 may be integrated.

One of the adhesive layers 170 is between the third conductive layer 123 and the readout layer 130. The adhesive layer 170 is pasted on the receiving layer 120 and on the readout layer 130. The other adhesive layer 170 is between the first conductive structure 113 and the second flexible circuit board layer 150. The adhesive layer 170 is pasted on first conductive structure 113 and on the second flexible circuit board layer 150. In at least one exemplary embodiment, the adhesive layer 170 is an anisotropic conductive film.

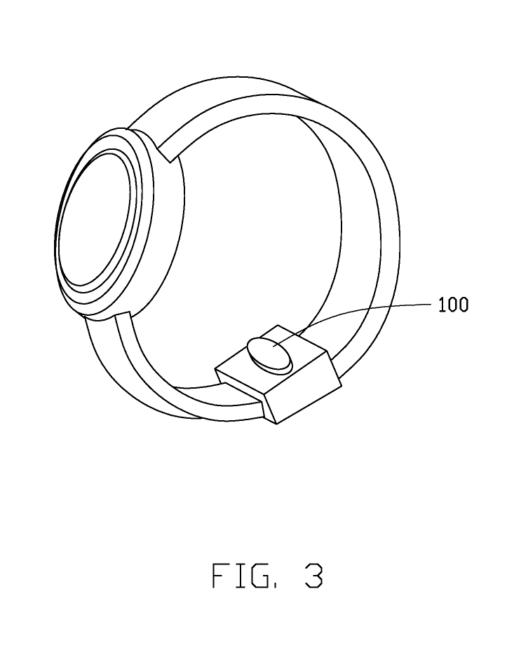

As shown in FIG. 3, the flexible sensing apparatus 100 is pasted or held against the specified object, such as a wrists. The voltage difference between the first conductive structure 113 and the second conductive structure 115 cause the transmitting element 111 to vibrate and generate ultrasonic waves, the ultrasonic waves pass through the receiving layer 120 and the third flexible circuit board 160 to reach the specified object. The receiving element 121 receives and converts the ultrasonic waves reflected into the localized electric charges based on the reflected ultrasonic waves. The third conductive layer 123 controls the localized electric charges to be transmitted to the readout layer by passing through the adhesive layer 170. The strength of the ultrasonic waves depends on a vasoconstriction in the specified object. The readout layer 130 converts the localized electric charges into electrical signals transmitted by the adhesive layer 170. The specified object may be in direct contact with the third flexible circuit board 160, or be spaced from the third flexible circuit board 160 for a specified distance.

By using flexible circuit board, flexibility and adhesion of the flexible sensing apparatus 100 are improved.

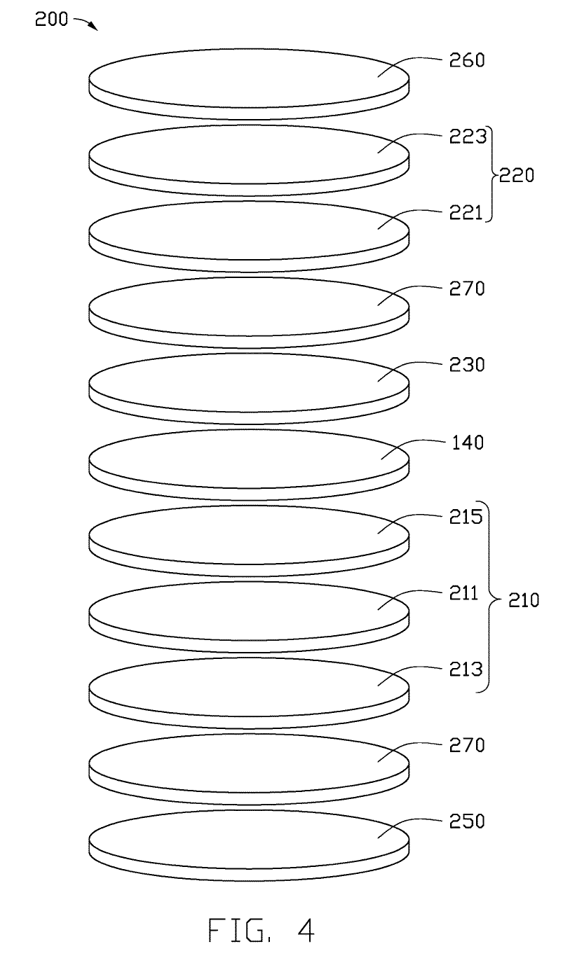

FIG. 4 illustrates a second exemplary embodiment of the flexible sensing apparatus 200. The flexible sensing apparatus 200 according to the second exemplary embodiment is approximately the same as the sensing apparatus 100. The differences between the flexible sensing apparatus 200 and the sensing apparatus 100 are hereinafter described.

The flexible sensing apparatus 200 includes a transmitting layer 210, a receiving layer 220, a readout layer 230, a first flexible circuit board 240, a second flexible circuit board 250, a third flexible circuit board 260, and two adhesive layers 270.

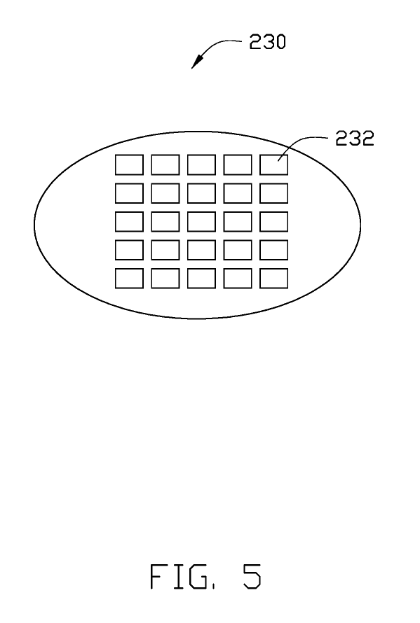

The readout layer 230 can convert the localized electric charges transmitted from the adhesive layer 270 into electrical signals, and transmit the electrical signals to an external circuit (not shown) for calculating a vital sign of the specified object. Referring to FIG. 5, the readout layer 230 includes a plurality of readout units 232 arranged in a matrix. The readout units 232 are driven by different signals in turn. Each of the readout units 232 includes a flexible thin film transistor (TFT) array, which receives the localized electric charges. The readout unit 232 is substantially rectangular. In at least one exemplary embodiment, the flexible TFT array can be high temperature polycrystalline TFT (HTPS-TFT), low temperature polycrystalline TFT (LTPS-TFT), amorphous silicon TFT (a-Si-TFT), or indium gallium zinc oxide TFT (IGZO TFT). In other exemplary embodiments, the readout unit 232 can be a triangle shape, an annular shape, or a polygon shape.

While various exemplary and preferred exemplary embodiments have been described, the disclosure is not limited thereto. On the contrary, various modifications and similar arrangements (as would be apparent to those skilled in the art) are intended to also be covered. Therefore, the scope of the appended claims should be accorded the broadest interpretation so as to encompass all such modifications and similar arrangements.

What is claimed is:

1. A flexible surface sensing apparatus pasted on a specified object for sensing vital signals of the specified object to convert into electrical signals, the sensing apparatus comprising:
a transmitting layer generating ultrasonic waves;
a receiving layer located on the transmitting layer, and the receiving layer receiving the ultrasonic waves reflected by the specified object, and converting the ultrasonic waves into localized electric charges; and
a readout layer between the transmitting layer and the receiving layer, and the readout layer converting the localized electric charges into electrical signals for representing a vital sign of the specified object;
wherein the receiving layer is between the specified object and the transmitting layer; the readout layer comprises a flexible thin film transistor (TFT) array; the flexible TFT array converts the localized electric charges generated by different positions of the receiving layer into the electrical signals.

2. The flexible surface sensing apparatus of claim 1, wherein the sensing apparatus further comprises a first flexible circuit board and a second flexible circuit board; the first flexible circuit board is between the transmitting layer and the readout layer, the first flexible circuit board provides a first specified voltage to the transmitting layer; the second flexible circuit board is on a surface of the transmitting layer facing away from the readout layer; the second flexible circuit board provides a second specified voltage to the transmitting layer.

3. The flexible surface sensing apparatus of claim 2, wherein the first flexible circuit board and the second flexible circuit board are insulated from each other.

4. The flexible surface sensing apparatus of claim 2, wherein the first flexible circuit board and the second flexible circuit board are electrically connected.

5. The flexible surface sensing apparatus of claim 1, wherein the readout layer comprises a plurality of readout units arranged in a matrix, each of the readout units comprises a TFT array; the readout units are driven by different signals in turn.

6. The flexible sensing apparatus of claim 1, wherein the flexible sensing apparatus is pasted on a cured or rough surface of the specified object.

7. The flexible surface sensing apparatus of claim 1, wherein the transmitting layer comprises a transmitting element, a first conductive structure, and a second conductive structure; the transmitting element is between the first conductive structure and the second conductive structure; the first conductive structure and the second conductive structure together drive the transmitting element to generate ultrasonic waves.

8. The flexible surface sensing apparatus of claim 7, further comprising an adhesive layer; wherein the adhesive layer is between the first conductive structure and the readout layer; the adhesive layer pastes the first conductive structure on the readout layer.

9. The flexible surface sensing apparatus of claim 7, further comprising an adhesive layer; wherein the adhesive layer is between the third conductive layer and the readout layer; the adhesive layer pastes the third conductive layer on the readout layer.

10. The flexible surface sensing apparatus of claim 9, wherein the receiving layer comprises a receiving element and a third conductive structure; the receiving element is between the third conductive structure and the readout layer; the conductive structure is between the receiving element and the third flexible circuit board; the third conductive structure provides a specified voltage; the receiving element converts the reflected ultrasonic waves into the localized electric charge, and the third conductive structure controls the localized electric charge to be transmitted to the readout layer.

11. A flexible surface sensing apparatus pasted on a specified object for sensing vital signals of the specified object to convert into electrical signals, the flexible sensing apparatus comprising:
a transmitting layer generating ultrasonic waves;
a receiving layer on a surface of the transmitting layer facing away from the readout layer, and the receiving layer receiving ultrasonic waves reflected by the specified object, and converting the ultrasonic waves into localized electric charges;
a readout layer between the transmitting layer and the receiving layer, and the readout layer converting the localized electric charges into electrical signals for representing a vital sign of the specified object;
wherein the receiving layer is located between the specified object and the transmitting layer; the transmitting layer comprises a first conductive structure, a second conductive structure, and a transmitting element between the first conductive structure and the second conductive structure; the first conductive structure receives the second specified voltage, and the second conductive structure receives the first specified voltage; the first conductive structure and the second conductive structure cooperate with each other to drive the transmitting element to generate ultrasonic waves.

12. The flexible surface sensing apparatus of claim 11, wherein the flexible sensing apparatus further comprises a first flexible circuit board and a second flexible circuit board; the first flexible circuit board is between the transmitting layer and the readout layer, the first flexible circuit board provides the first specified voltage to the transmitting layer; the second flexible circuit board is located on a surface of the transmitting layer facing away from the readout layer; the second flexible circuit board provides the second specified voltage to the transmitting layer.

13. The flexible surface sensing apparatus of claim 12, wherein the receiving layer comprises a receiving element and a third conductive structure; the receiving element is between the third conductive structure and the readout layer; the conductive structure is between the receiving element and the third flexible circuit board; the receiving element converts the reflected ultrasonic waves into the localized electric charges, and the third conductive structure controls localized electric charges to be transmitted to the readout layer.

14. The flexible surface sensing apparatus of claim 12, wherein the first flexible circuit board and the second flexible circuit board are insulated from each other.

15. The flexible surface sensing apparatus of claim 12, wherein the first flexible circuit board and the second flexible circuit board are electrically connected.

16. The flexible surface sensing apparatus of claim 11, wherein the readout layer comprises a one-piece readout unit; the readout unit comprises a flexible thin film transistor (TFT) array; the flexible TFT array converts the localized electric charges generated by different positions of the receiving layer into the electrical signals.

17. The flexible surface sensing apparatus of claim 11, wherein the readout layer comprises a plurality of readout units arranged in a matrix, each of the readout units comprises a flexible thin film transistor (TFT) array, the readout units are driven by different signals in turn.

18. The flexible surface sensing apparatus of claim 11, wherein the flexible sensing apparatus pastes on a cured or rough surface of the specified object.

19. The flexible surface sensing apparatus of claim 11, further comprising an adhesive layer; wherein the adhesive layer is between the first conductive structure and the readout layer; the adhesive layer pastes the first conductive structure on the readout layer.

20. The flexible surface sensing apparatus of claim 11, further comprising an adhesive layer; wherein the adhesive layer is between the third conductive layer and the readout layer; the adhesive layer pastes the third conductive layer on the readout layer.
